(19) United States
(12) Patent Application Publication
(10) Pub. No.: US 2013/0156858 A1
(43) Pub. Date: Jun. 20, 2013

(54) Formulations Preserving Bioactivity and Methods of Their Preparation

(71) Applicants: Per Wikström, Upplands Vasby (SE); Tobias Bramer, Goteborg (SE); Lilyan Yachoh, Sodertalje (SE)

(72) Inventors: Per Wikström, Upplands Vasby (SE); Tobias Bramer, Goteborg (SE); Lilyan Yachoh, Sodertalje (SE)

(73) Assignee: RPH Pharmaceuticals AB, Haninge (SE)

(21) Appl. No.: 13/701,579

(22) PCT Filed: May 30, 2011
(86) PCT No.: PCT/SE2011/050670

§ 371 Date: Feb. 28, 2013

(30) Foreign Application Priority Data
Jun. 3, 2010 (SE) 1050566-7

(51) Int. Cl. A61K 9/50

(52) U.S. Cl. CPC A61K 9/5036; A61K 9/5089
USPC 424/493; 424/490; 424/497; 424/94.6

(57) ABSTRACT

A pharmaceutical composition comprising an alginate hydrogel core where a bioactive agent is entrapped. The water content of the hydrogel is at least 10% of equilibrium water content. The beads have an enteric coating and are intended for oral administration. The bioactive agent is bioactive proteins, antibodies or viable cells and it is intended to exert its activity in the duodenum and the upper intestines.

### FIELD OF INVENTION

The present invention relates to a method of manufacturing coated hydrogel beads while preventing loss of water from the hydrogel. The invention also relate to enterically coated beads of a hydrogel suitable for delivery of sensitive biological systems to the upper intestines.

### BACKGROUND OF THE INVENTION

Capsulation of bioactive compounds with calcium alginate and a polycation is well described in the scientific literature. Chitosan and polylysine are the most commonly used polycations for capsule production, but DEAE-dextran has also been utilized. Alginate and chitosan have the ability to combine in a complex, that change the pore-size that are able to act as a sieve enclosing larger protein but letting smaller molecules through. The permeability of the walls of chitosan-alginate capsules is adjusted by substituting with esterified alginic acid a portion of the metal alginate normally used in the fabrication of such capsules, see U.S. Pat. No. 4,808,707. A system of swollen alginate-chitosan beads entrapping allyl isothiocyate was studied by Kim et al. in Carbohydrate Polymers, 2008, 71, 566-573. However, this article does not advice further on how such a system can be further adapted to orally deliver liable protein based bioactive agents through the gastric levels to the upper intestines.

In US Patent Application published as 2006/0228422 the combination alginate chitosan-Ca2⁺ is employed to produce microcapsules and carriers aiming at producing systems for oral administration of biologically active substances protected from degradation at the gastric level, allowing release at the intestinal level. In this application it is described that the beads formed are thoroughly dried (at 24 h at 37° or spray-dried) in order to remove water. However, due to the drying procedure, it is a risk that the beads with the active biological substance collapse in its gel-structure. This fact may compromise the efficacy of the bioactive agent to exert its activity in the intestinal part of the gastric system. Further, Indian J of Pharm Sci, 2010, 7281), 18-23 (MS Khan et al) discloses alginate and chitosan hydrogel beads having an enteric coating of Eudragit S100 for colonic delivery of theophylline. However, this article leaves no particular provisions how to adapt the beads to establish a protective environment throughout productions steps, storage and administration to preserve the activity of liable protein based active agent such as enzymes.

It is evident that there is need for new systems for oral administration of biologically active substances, suitable to safely and efficiently deliver bioactive agents to the intestines, in natural polysaccharide beads which have the characteristics of: i) entrapping at least one biologically active substance so that it is protected from degradation in acidic environment at gastric level and ii) admitting desired effective bioactivity.

Additionally, such new systems shall be capable of retaining or preserving water in the beads while at the same time protecting the entrapped biologically active substance from degradation in acidic environment of the stomach.

### DESCRIPTION OF THE INVENTION

In general terms the invention relates to a pharmaceutical composition for oral or enteral administration comprising beads that are targeted to exert the bioactivity in the duodenum and the upper intestines. The beads typically comprises such bioactive agents (proteins, antibodies or viable cells) which are liable at a low pH in the stomach cavity, proteolytic degradation in the duodenum and addition may need a controlled water content in the beads in order to maintain activity throughout manufacturing and storage processes. For this reason, the beads comprise at least one ionically cross-linked hydrogel core, preferably comprising alginate, the bioactive agent entrapped in the hydrogel gel pores, an enteric coating that is water soluble above a threshold pH value between 4 and 7, while the water content of the hydrogel is at least 10% of its equilibrium water content.

Maintaining the water content of the hydrogel core represents an important part of the invention, as it is fundamental for maintaining the structure of the beads which otherwise may collapse or crack thereby risking that composition is impaired or unreliable as a safe carrier for a bioactive agent with a suitably controlled efficacy in the duodenum. A controlled water content is also necessary in order to preserve the activity of the bioactive agent such as proteins and cells during manufacturing, storage and administration; while the bioactive agent rapidly exerts its activity in the duodenum due to the preserved bead structure.

Preferably, the water content of the hydrogel core is between 20 to 98%, more preferably, between 50 to 98%, and even more preferably between 70 to 98% of equilibrium water content. The bioactive agent can be physically entrapped in the hydrogel, which means that the crosslinked structure cages the high molecular weight bioactive agent. It is also conceivable to use other well know immobilization techniques including conjugation with chemical or physical bonds. The hydrogel can be formed from natural or synthetic water-insoluble, hydrophilic crosslinked polymer chains, preferably originating from alginate. The equilibrium water content of the hydrogel is controlled both by the structure of the hydrogel and its crosslink density.

The enteric coating preferably is acylate based, more preferably, the enteric coating comprises a copolymer of methacrylic acid and an acrylate. One suitable coating comprises methacrylic acid and ethylacrylate. A suitable such brand for water based systems is marketed as Eudragit L 30 D-55, while Eudragit L 100-55 is suitable in systems with ethanol as a solvent (both from Röhm Pharma Polymers). However, the skilled artisan can find suitable alternatives of coatings that can withstand the gastric fluids and become soluble in the duodenum to be useful for such a controlled release of a bioactive agent.

According to another aspect of the invention, the beads have a gel layer between the hydrogel core and the coating. It is preferred that this gel layer has pores of smaller average size than the average size of the pores of the hydrogel. The additional gel layer can be designed to contribute to the entrapment of the bioactive agent while it exerts its activity in the duodenum and lower parts of the gastric system, while it also can be designed to prevent degradation and/or inactivation of the bioactive agent from enzymes present in the fluids of the gastric system.

Preferably, the hydrogel cores comprises alginate and are surrounded with a gel layer. Alginate is well-known agent that crosslinks to a hydrogel in the presence of a divalent cation, such as calcium. It is conceivable that both natural and derivatized alginate is useful with the present invention, as long as its functionality as a hydrogel is retained. The skilled person is also knowledgeable of how to modify the pore size of such alginates and how to accomplish different sizes of alginate cores, such as in the range 0.001 to 5 mm, as contemplated with embodiments of the present compositions.

The gel layer preferably comprises chitosan or functional derivatives of chitosan which also is a well-known gel-forming substance. A useful alternative to chitosan is polylysine. Preferably, the gel layer is formed from a mixture of chitosan and alginate which is crosslinked together in a conventional manner. The chitosan comprising layer is advantageously maintained as a shell when the beads are transported through the gastrointestinal system. Preferably, the pores of the gel layer have a smaller average size than the pores of the alginate core. It is important that the gel layer pores are sufficiently small to prevent proteolytic enzymes from degrading the entrapped bioactive agent.

In one embodiment, the inventive compositions relates to a plurality of enterically coated beads have an average size in the range of 0.1 to 2 mm with alginate cores with entrapped bioactive agent having a surrounding layer comprising alginate and chitosan, wherein the beads have an average size in the range of 0.1 to 2 mm. The beads can be further collected in a dose form suitable for oral or enteral administration.

In another embodiment, the inventive compositions relate to a plurality of enterically coated beads have an average size in the range of 0.1 to 2 mm, wherein the beads are made of gelatin including a plurality of alginate cores (microbeads) each with entrapped bioactive agent having a surrounding layer comprising alginate and chitosan, wherein the cores have an average size in the range of 10 to 100 μm. The beads can be further collected in a dose form suitable for oral or enteral administration.

The invention also generally relates to a method of manufacturing the previously described bead compositions. comprising a) an ionically cross-linked hydrogel, b) a bioactive agent entrapped in the gel pores. The method comprises the steps of:

preparing cores in a solution comprising alginate in the presence of a crosslinking ion and the bioactive active agent; treating the cores in order to control their water content and structure to thereby settling the bioactive beads; fluidizing the beads in a fluidized bed; feeding a composition comprising an enteric coating agent to the fluidized bed; and collecting the so prepared beads for the preparation of an orally or enterally administrable pharmaceutical composition.

In one embodiment, the method further comprises a step of separating the cores from the solution, and includes allowing the cores to dry to a controlled water content of at least 10% of the equilibrium water content of the hydrogel, preferably between 20 to 98%, more preferably, between 50 to 98%, still more preferably between 70 to 98% of equilibrium water content. Alternatively described, in order to prepare the cores for fluidization, they are dried to weigh loss of about 5 to 10% (wt) which generate surfaces that are sufficiently dry to avoid agglomeration, but preserve sufficient amount of water to safeguard the bioactive agent. Preferably, the drying process in step is performed at ambient temperature for at least one hour, more preferably about two hours.

In another embodiment, the method comprises preparing the cores in a water-in-oil microemulsion and the treatment step includes admixing gelatin with the solution of the preparation step at a temperature allowing gelatin to be melted, thereby forming beads including the cores at a lower temperature and separating the beads. Preferably, the solution further comprises chitosan in order to prepare a gel layer of alginate and chitosan surrounding an alginate core with entrapped bioactive agent.

In still another embodiment, the treatment step includes contacting the cores with a detackifier, such as magnesium stearate or non-crosslinked chitosan. Preferably, the enteric coating agent is a solution based on ethanol.

The coating substance is preferably an enteric coating substance as earlier described which is fed to the fluidized cores by a spraying process conventional in pharmaceutical manufacturing. The coating substance preferably is a suspension that forms drops on bead surfaces which subsequently coalesce into a covering coating.

The method can also when suitable comprise a step of precoating the hydrogel cores with a gel component. This gel component provides an outer layer on the hydrogel core with less average pore size than the average pore size of the hydrogel cores. The precoating step is performed before the fluidizing step. Preferably, the precoating step takes place in the preparatory step by including the gel component in the solution. Alternatively, the precoating step can be performed by collecting the cores from their preparatory solution and subjecting them to a solution comprising the gel component. The formation of the hydrogel and the outer layer is performed with materials and methods as earlier described. According to one useful embodiment the hydrogel cores are formed from alginate crosslinked with calcium ions and the gel component is formed from crosslinking alginate and chitosan. The producing step involves a solution comprising chitosan and calcium ions to which a solution comprising alginate and a bioactive agent is supplied in controlled form. Hydrogel beads with a crosslinked alginate core having an entrapped bioactive agent is formed, which has a gel layer of chitosan/alginate as an outer shell. The resulting beads are collected and coated with the earlier described method steps, thereby enabling controlled water content in the hydrogel core. The chitosan has a very low solubility in the duodenum and also further down the digestive tract. This makes the beads stable in this environment, when forming complex with alginate in duodenum and in the small intestine. The coating will prevent the beads to shrink by losing water and preserve the integrity of the beads in such way that a smaller bioactive agents stay immobilized and prevented proteolytic degradation (trypsin, chymotrypsin and elastase) in the duodenum when the coating is removed

The beads according to the invention and the manufacturing process with their design that safeguards efficacy of liable bioactive components during their delivered to the duodenum open up a number of therapeutic possibilities.

Accordingly, the present invention generally refers to treatment methods comprising intraduodenally inactivating a substance excreted to the duodenum by orally or enterally administering compositions of the previously described beads. In such a therapy, the bioactive agent entrapped in the aqueous environment, preferably is an enzyme that directly in the duodenum, enzymatically inactivates such a substance that is unwanted and potentially harmful.

In a special such embodiment, the bioactive agent is beta-lactamase capable of inactivating an antibiotic excreted to the duodenum of a patient that is subjected to an intravenous treatment with an antibiotic. Such an additional treatment is of great benefit for critical care patients dependent on treatment for massive infections and considerable risks to acquire secondary gastric complications.

In another embodiment, the bioactive agent is are cells entrapped in the hydrogel beads, capable of producing lactase, suitable for a therapy of treating lactose intolerance by degrading lactose in the duodenum and the following intestines.

In another embodiment antibodies with specifically bind to proteins or protein-fragments involved in celiac disease or enzymes capable of hydrolyzing such fragments are entrapped. Because pepsin acts in the stomach and trypsin, chymotrypsin and elastase act in the duodenum, there will most likely be smaller fragments of the proteins involved in celiac disease. These fragments will in the pathological process be substrate to tTG that will turn the fragments that will trigger the T-cells and the following pathological events causing the disease. The present invention is able to prevent the disease causing fragments to enter the system and be removed in feces.

The compositions of the present invention can be useful for a number of therapies when a liable bioactive agent is needed to be delivered in active form to the duodenum.

### DETAILED AND EXEMPLIFYING DESCRIPTION OF THE INVENTION

Other features and uses of the invention and their associated advantages will be evident to a person skilled in the art upon reading the description and the examples.

It is to be understood that this invention is not limited to the particular embodiments shown here. The following examples are provided for illustrative purposes and are not intended to limit the scope of the invention since the scope of the present invention is limited only by the appended claims and equivalents thereof.

If nothing else is defined, any terms and scientific terminology used herein are intended to have the meanings commonly understood by those of skill in the art to which this invention pertains.

The term “about” as used in connection with a numerical value throughout the description and the claims denotes an interval of accuracy, familiar and acceptable to a person skilled in the art. Said interval is ±10%.

In the following experimental part of the description, alginate-chitosan-Ca2⁺ formed beads that are coated in a fluidized bed. The coating procedure is preserving water in the beads. Beads are produced that protect biologically active agent from the gastric low pH, but then release its protecting coat and with its immobilized biological activity remove undesired substances in the intestinal environment. The experiments demonstrated that immobilized enzyme (Candida rugosa lipase) in the beads where unable to cope with the acidic conditions in the stomach unless the beads enterally coated. The beads are manufactured with purposely preserved content of water by coating the beads with enteric coat that will entrap the water as well as protect the biological activity from pH degradation. Further the chitosan polymer has a high solubility in the acidic environment in the stomach, but when entering the duodenum pH will be approximately 6 and chitosan has a very poor solubility at this pH. In the given example with immobilized lipase the beads were manufactured at pH 4 and then increased to neutral, because of the solubility of chitosan at lower pH. The fact that chitosan has a very low solubility in the duodenum and also further down the digestive tract is contributory to the efficacy of the invention.

### BRIEF DESCRIPTION OF DRAWINGS

The invention is now described, by way of example, with reference to the accompanying drawings, in which:

FIG. 1 shows the pH influence on free lipase activity.

FIG. 2 shows the pH influence on lipase activity measured after dissolution of uncoated untreated or empty beads, respectively.

FIG. 3 shows the lipase activity in uncoated gel beads treated or untreated with alcalase, respectively.

FIG. 4 shows the lipase activity in uncoated gel beads treated with different gastric conditions and ± alcalase.

FIG. 5 shows an image of beads treated with enteric coating in aqueous phase.

FIG. 6 shows an image of beads in different preparative stages and a dried bead as comparison.

FIG. 7 shows an image of beads in different preparative stages including pretreatment and coating.

### EXAMPLES

### 1. Materials and Methods

### 1.1 Materials

Chitosan and sodium alginate were bought from Sigma, St Louis, USA. Candida rugosa lipase (Sigma L1754, 724 U/mg of protein, St Louis, USA) was used as the model enzyme. The substrate was 1.2-Di-O-lauryl-rac-glycero-3-glutaric acid 6′-methylresorufinester (DGGR), (Sigma, St Louis, USA). Proteolytical enzymes were alcalase 2.4L FG (Novozyme Bagsvaerd, Denmark). The Lactase was from Aspergillus oryzae manufactured by Oy Verman Ab, Kerava, Finland. The substrate o-Nitrophenyl β-D-galactopyranoside (ONPG) from Sigma; Sigma N1127 Hemoglobin from bovine blood (Sigma, St Louis, USA) was used. Eudragit® L 30 D-55 was the enteric coat. All other materials were taken from local suppliers and distilled water was used in all preparations.

### 1.2 Methods

### 1.2.1 Lipase Activity Measurements After Exposure to Various pH Values.

To investigate if the model enzyme would preserve its structure and activity during the immobilizing procedure, initial experiments were made to measure lipase activity after exposing the enzyme to pH 4 for the corresponding time period used during immobilization. The lower pH values during the immobilization phase were needed due to the low solubility of chitosan at higher pH. Chitosan with lower molecular weight and solubility at higher pH is conceivable to use for pH-sensitive biomolecules.

### 1.2.2 Preparation of Alginate-Chitosan Gel Beads

2% Sodium alginate solution containing the enzyme lipase (10 mg/ml) was filled in a syringe with a needle and dropped directly in a buffer-solution (pH 4) containing 0.25% chitosan and 0.25% acetic acid. When alginate is dropped in a chitosan solution, a shell of alginate-chitosan complex is formed; the complex formation is strong due to the multiple numbers of charged groups. However, the beads need to be further stabilized and calcium ions were added to the chitosan solution (50 mM CaCl₂) to gellify the core. Ca2⁺ ions will diffuse in to the alginate core. The beads were stored in 0.1 M MES buffer solution of pH 6. To visually ensure encapsulation without leakage, an experiment to encapsulate hemoglobin from bovine blood (brown colored) was initially preformed.

### 1.2.3 Gel-bead Evaluation of Enzymatic Activity

Beads were dissolved by adding 0.5 g beads to 3 ml 67 mM EDTA, 0.33 M NaCl pH 6.0, while stirring (IKA® RCT basic, Staufen Germany) for about 20 min, followed by filtration with Millex® GV 0.22 μm. Dissolving of the beads was necessary to measure total enzyme activity during the preparation, storage and final simulation studies.

1.2.4 Influence of Exposure to Different pH on Immobilized (uncoated) Lipase Activity

The gel beads were exposed to different pH-conditions: 0.1 M glycine buffer of pH 2; 0.1 M acetate buffer of pH 4; 0.1 M MES buffer of pH 6; 0.1 M Tris-HCl buffer of pH 8 for 30 min, to evaluate any leakage from the beads or inhibition of enzyme activity. The beads were then washed, dissolved and the retained activity was measured in a spectrophotometer. This experiment was preformed to evaluate the treatment's influence on the enzyme activity.

### 1.2.5 Lipase Activity

The Lipase activity was measured by using a substrate solution containing DGGR (0.24 mM), 7.2 mM Tautrodesoxycholate and 1.6 mM tartrate buffer pH 4.0 diluted in 41 mM tris-HCl pH 8.4 and sample (beads prepared or not prepared, respectively). DGGR (750 Da) gives a color change from yellow to reddish when transformed to the products gluteric acid and metylresorfin. The formation of metylresorfin is directly proportional to the enzyme activity and the increased absorbance was measured with a spectrophotometer 8453 UV-visible spectrophotometer (Agilent Technologies, Waldbronn, Germany) with a diode array detector (Agilent Technologies, Waldbronn, Germany), at the wavelength 580 nm. Activity was measured according to the method described above. There was no substrate limitation in all the activity measurements.

### 1.2.6 Lipase Degradation Using Alcalase as Proteolytic Enzyme

Alcalase has a molecular weight of 27,300 Da, which is close to pancreatic trypsin (24,000 Da) and chymotrypsin (25,000 Da). Therefore alcalase was a suitable proteolytic model enzyme for this study, imitating human proteolytical enzymes.

To determine the appropriate amount of alcalase needed to effectively degrade lipase, an experiment was performed on free lipase solution with different quantities of alcalase in a 0.1 M MES buffer of pH 6.0 for 30 min. The remaining lipase activity was determined.

The effect on immobilized lipase in uncoated beads was also investigated, by treatment with alcalase for 1 h in a 0.1 M MES buffer of pH 6.0. Beads were then dissolved and lipase activity was measured.

### 1.2.7 Enteric Coating of Beads.

For designing the gel beads to have the duodenum as a target, the gel beads was coated with Eudragit® in a Strea-1 fluid bed dryer (GEA Pharma Systems, Eastleigh, UK). The layer Eudragit® coating the beads was 4 mg/cm². Eudragit® L 30 D-55 dissolves at a pH above 5.5, with a release site in the duodenum (Evonik Industries, 2008).

1.2.8 Challenging Enteric Coated Gel Beads with Low pH.

Composite-beads with immobilized lipase coated with Eudragit® were placed in a 0.1 M glycine buffer solution of pH 1.2 for 3h while stirring (IKA® RCT basic, Staufen Germany) withdrawals of beads were made after 1 h and 3 h, and activity was determined after dissolving of beads. After 3 h the remaining beads were washed and divided in two different 0.1 M MES buffer solutions of pH 6.0 for 1 h, where one of the solutions contained alcalase. After 1 h the lipase activity was determined with both intact beads and dissolved beads.

### 1.3 Statistics

The enzyme activity measurements in the final experiments were performed in triplicates and the results was based on mean values, and standard deviations.

### 2. Results

### 2.1 Lipase Activity Measurements After Exposure to Various pH Values.

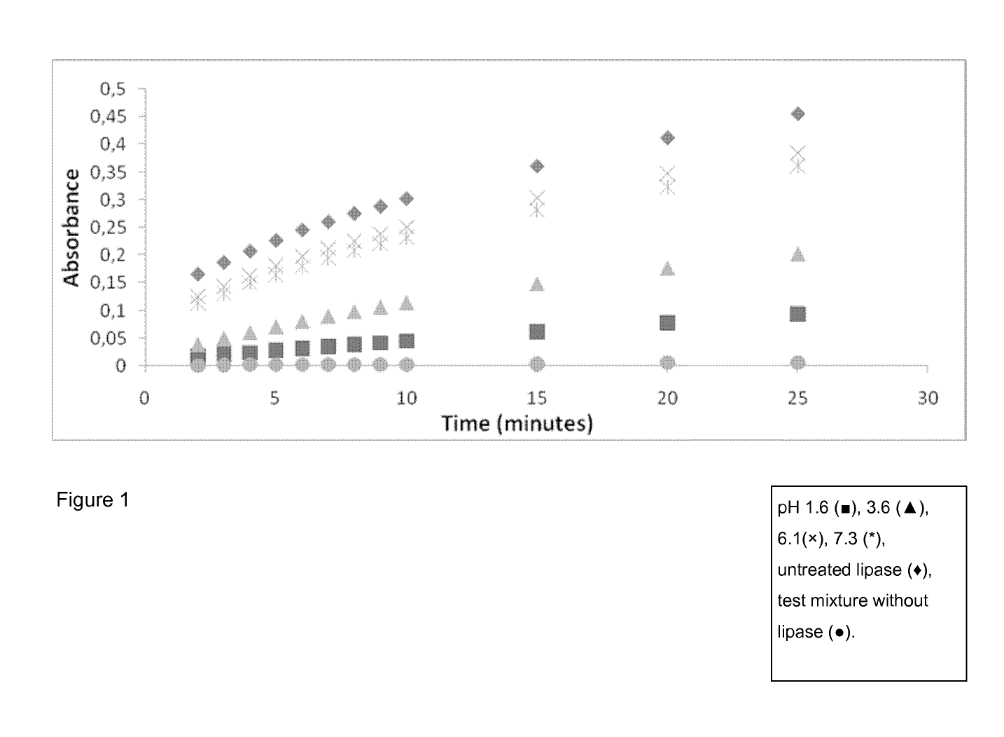

FIG. 1 show clearly that the lipase activity was inhibited at lower pH values, wherein the different pH values used in the exposures are symbolized by: pH 1.6 (□), 3.6 (▴), 6.1(X), 7.3 (*), untreated lipase (⋄), test mixture without lipase (). Activity was measured during 30 minutes for all samples. Even at pH 3.6 which is close to the pH needed for preparation of the beads, the lipase activity was inhibited.

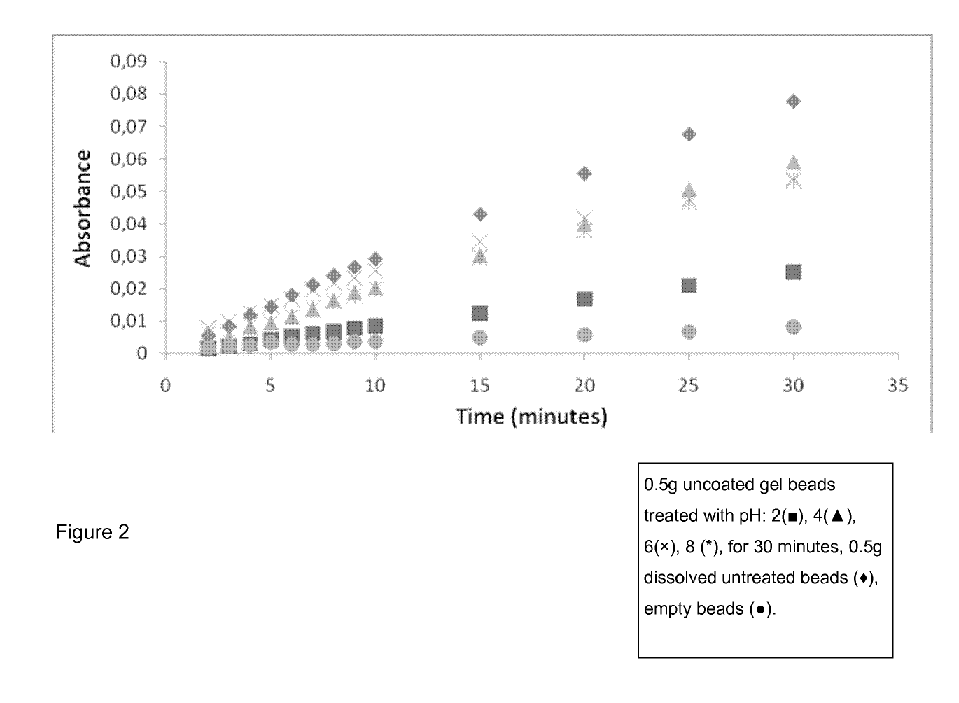

The pH influence on immobilized uncoated beads (FIG. 2) was similar to those in solution. Lipase (approximately 12.3 μg/ml) activity was measured after dissolution of 0.5 g uncoated gel beads treated with pH: 2(□), 4(▴), 6(X), 8 (*), for 30 minutes, 0.5 g dissolved untreated beads (⋄), empty beads (). Activity was measured during 30 minutes for all samples. The activity measurements with lipase solution showed a greater difference between lipase exposed to pH 3.6 and 6.1, but with the immobilized lipase beads there was no difference.

2.2 Gel beads encapsulation efficacy.

The highest concentration of lipase encapsulated in the beads without loss of bead strength was 10 mg/ml. Other concentration gave either to weak beads or unsatisfying activity after dissolution.

The activity for known concentrations of lipase was measured and used as comparison to how much lipase was lost during the preparation of the beads. The theoretical amount of lipase loaded was 10 mg/ml, after dissolved, concentration of the solution should have been 23.4 μg/ml. Dissolved beads showed an activity more close to the activity of 12.3 μg/ml indicating a loading efficacy of almost 53%.

### 4.3 Lipase Degradation Using Alcalase as Proteolytic Enzyme

The experiment with alcalase and free lipase showed that alcalase inhibited lipase and the smallest quantity of alcalase that gave complete inhibition was chosen for the further experiments, which was 10 times less alcalase than buffer.

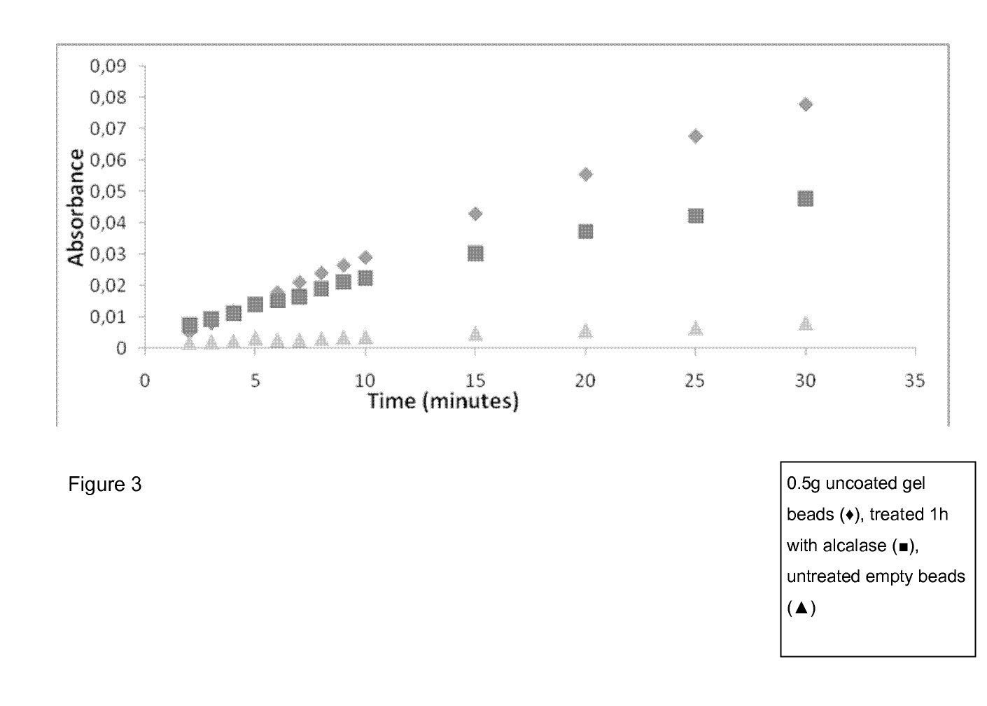

The lipase (approximately 12.3 μg/ml) activity measured after dissolving of 0.5 g uncoated gel beads (⋄), treated 1 h with alcalase (□), untreated empty beads (▴) is shown in FIG. 3. The activity was measured during 30 min for all samples.

However, with the immobilized lipase beads, after 1 h in alcalase the lipase activity had decreased, but more than 50% of activity still remained (FIG. 3), compared to the free lipase where the activity was inhibited fully when exposed to alcalase for 30 min. This suggests that the beads have the ability to protect the lipase from proteolytical degradation.

### 4.4 Enteric Coating of Beads

It is of importance to retain an adequate water content in the beads in order to maintain the bead structure and thereby it is predetermined porosity to prevent the bioactive agent (such as an enzyme) to migrate and to prevent the bioactive agent from proteolytic activity in the duodenum. At the same time, in order to be correctly enterally coated in a fluidized bed, the beads must be correctly fluidized without aggregating as may be the result if the bead surface is sticky. The following examples relate to different methodologies of counteracting this problem

### 4.4.1 Enteric Coating in an Aqueous Phase

Beads made in accordance with paragraph 1.2.2 above were treated with a partial drying process in room temperature for 2 hours to obtain a reduction in weight of less than 7%. The so treated beads were fluidized and subjected to spray coating with

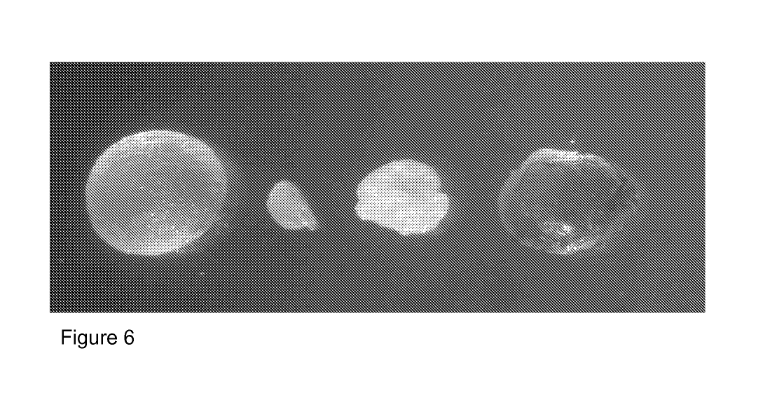

Eudragit L30 D-55 suspended in aqueous solution. Referring to FIG. 6 which shows from left to right; a beads before coating with Eudragit suspended in water; a bead after pre-drying; a bead coated with Eudragit; and a bead with Eudragit removed at a pH>5.5. As a result, the beads can be coated without losing substantial water content and thereby destroyed pore structure or size, which is of importance for the integrity of an entrapped bioactive agent.

4.4.2 Enteric Coating with Eudragit Dissolved in Ethanol

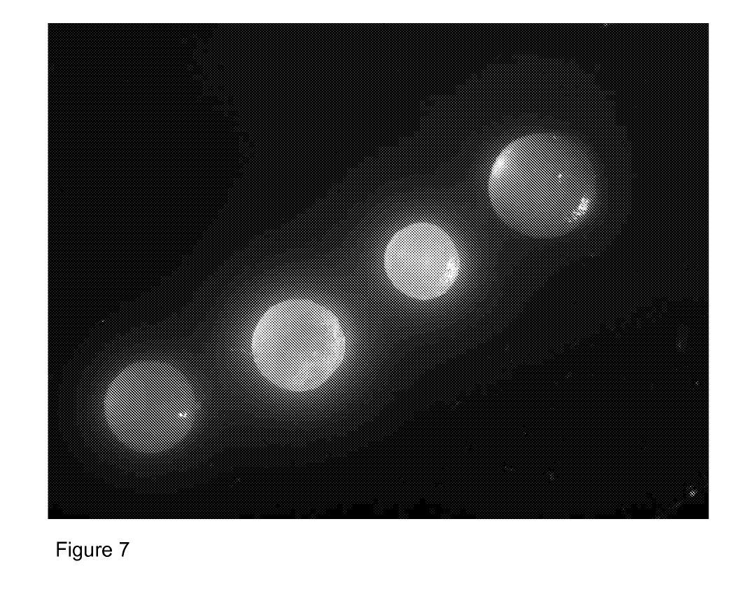

Beads made in accordance with paragraph 1.2.2 above were treated by powdering with magnesium stearate. The powdering process was performed by collecting and gently shaking the beads in a sieve, whereupon the beads were transferred to a larger plastic container and shaken together with dry, powdery magnesium stearate. Before transferration to a fluidized bed, excess powder was removed. The so prepared beads were coated with 62.5 g Eudragit L 100-55, 6.25 g triethyl citrate, 18.75 g magnesium stearate, 866.8 g ethanol and 45.6 g water. The beads were readily fluidized and spray coated at 25° C. without aggregation and with the water content retained. FIG. 7 shows from left to right; a bead before treatment; a bead following powdering with magnesium stearate; a bead following coating; and a bead with coating removed at a pH>5,5. FIG. 7 demonstrates that he beads maintain their structure (with an acceptable water loss), even more so than with the aqueous process. This process can successfully be repeated with dry powdery chitosan, but using talc led to aggregation in the fluidized bed.

In another alternative to this process, the beads are wetted with gelatin before powdering with magnesium stearate or chitosan. As a result, beads with excellent fluidizing capacity are obtained which have a complementary layer for safe-guarding from loss of water content during the process. The hardness of the gelatin can be controlled by the percentage gelatin in the wetting solution.

4.5 Beads with entrapped lactase

Beads were prepared in accordance with paragraphs 1.1 and 1.2, above, but with lactase replacing lipase. The beads were treated in four different ways: (a) storage in room temperature over night in 0.1 M MES buffer pH 6 followed by washing with 3×5 ml buffer and addition of ONPG for determination of enzymatic activity; (b) drying over night at room temperature and reconstitution with 0.1 M MES buffer followed by washing with 3×5 ml buffer and addition of ONPG for determination of enzymatic activity; (c) storage in room temperature over night in 0.1 M MES buffer pH 6 followed by washing with 3×5 ml buffer, treatment with proteolytic enzyme (alcalase) and addition of ONPG for determination of enzymatic activity; and (d) drying over night at room temperature and reconstitution with 0.1 M MES buffer followed by washing with 3×5 ml buffer, treatment with proteolytic enzyme (alcalase) and addition of ONPG for determination of enzymatic activity.

The tests demonstrated drying of the beads resulted in fragmentation and changed form after reconstitution. There was no difference in lactase activity in dried beads, however, in dried, reconstituted, beads treated with proteolytic enzyme (alcase, similar in size to such enzymes prevalent in the duodenum) lactase was effectively degraded. It is concluded that retaining the water content in the beads throughout the manufacturing process is of critical importance to maintain its size-exclusing capacity. Should the bead structure be impaired during the manufacturing, the entrapped bioactive agent will rapidly become degraded of the proteolytic enzymes in the duodenum (such as trypsin, chymotrypsin and elastase), resulting in disappearance of the desired bioactive effect.

4.6 Challenging Enteric Coated Gel Beads with Low pH.

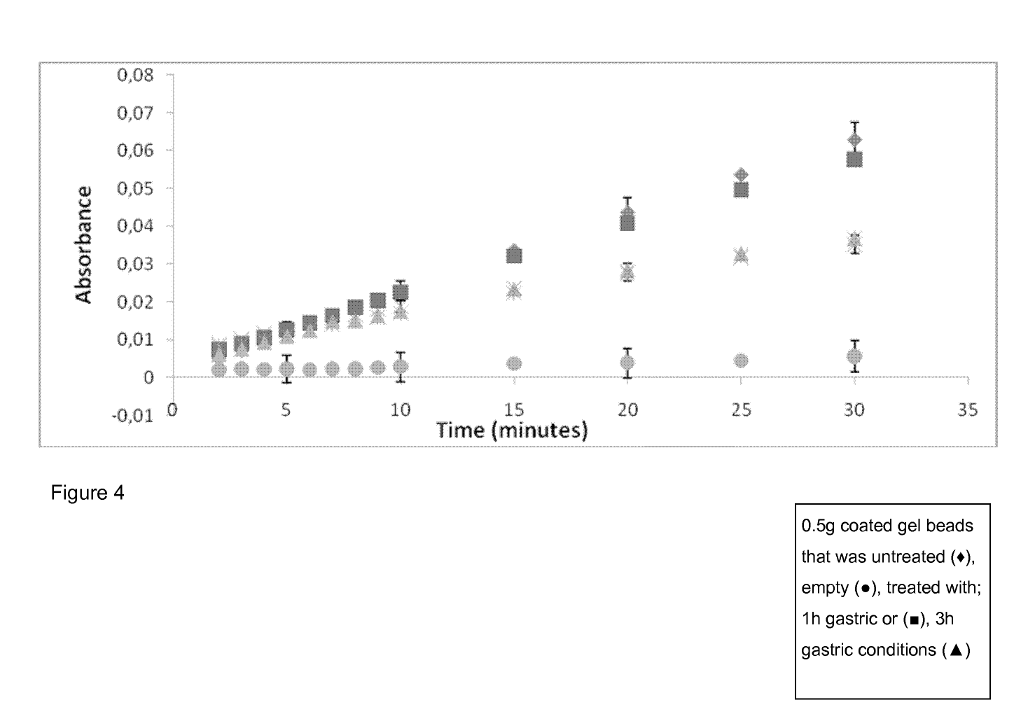

After 3 h in acidic buffer solution the enteric coating of the beads protected the lipase to a large extent from being inactivated (FIG. 4). Immobilized lipase (approximately 12.3 μg/ml) activity was measured after dissolution of 0.5 g coated gel beads that was: untreated (⋄), empty (), treated with; 1 h gastric or (□), 3 h gastric conditions (A), respectively. Remained beads after 3 h in gastric conditions, treated with 1 h duodenal conditions with alcalase (X), 1 h duodenal conditions without alcalase (*). Activity was measured during 30 min for all samples. Following the low pH treatment, the coated beads were treated for 1 h at pH 6.0 with or without alcalase exposure. There was no difference between the alcalase exposed and unexposed beads when comparing their lipase activity as shown in FIG. 4. For uncoated beads exposed to alcalase there was some decrease in activity after the exposure (See FIG. 3).

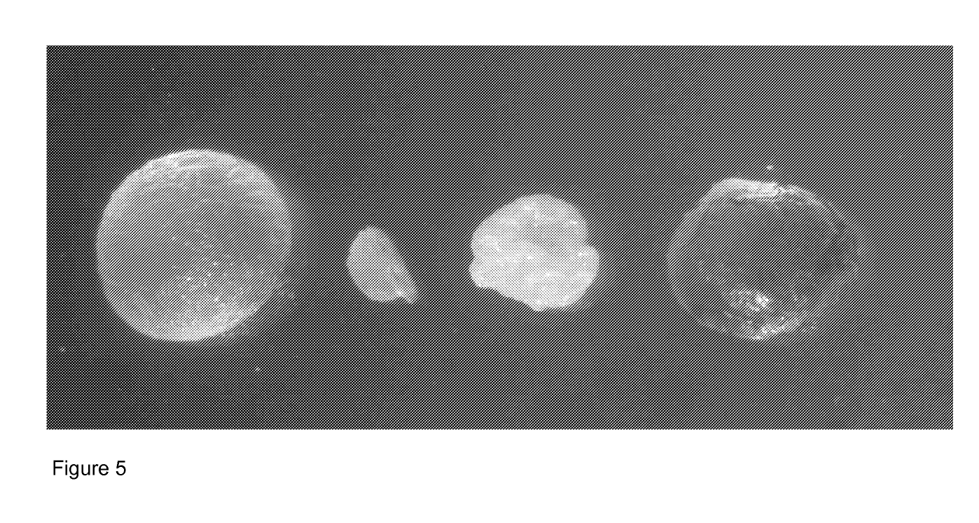

After 3 h in pH 1.2 and 1 h in pH 6.0, the activity was measured in whole beads as described above. Activity remained and beads where still intact and also reverted to their spherical shape after being shrunken during the coating as shown in FIG. 5. The image Is taken by an Olympus SZH stereozoom microscope, Japan, shows from the left: an uncoated bead, a dried uncoated bead, an Eudragit® coated bead and an Eudragit® coated bead after dissolving of the enteric coat.

Dried uncoated beads were smallest and contained no water, the coated beads however. entrapped water during the coating process and was further rehydrated when the enteric coat was removed.

The inventors have shown with this study that it is possible to immobilize enzyme with remaining activity in composite gel beads. The beads can be entirely coated with a portion of water remaining in the beads. There is only some inactivation of enzyme activity at low pH mimicking the conditions in the stomach, but the beads are still enzymatically active. After the enteric coat was dissolved in higher pH values with the presence of proteolytic enzyme, the beads remained intact and the enzyme was still active, showing that the bead provided a steric obstacle for the proteolytic enzyme (27,300 Da) but allowed for diffusion of the substrate (750 Da).

The inventors have successfully designed a therapeutically active bead, comprising one or a plurality of therapeutically active agents or molecules, with the duodenum as the target organ. Moreover, the disclosed invention can be used for designing therapeutically active beads with other target regions, depending on the type of Eudragit® used.

4.7 Beads with Microsized Cores Prepared with Emulsion Technology

As an alternative to the above described beads made with a drop technology microbeads are prepared and treated with gelatin to form beads in the size of 1 to 2 mm.

A first container was provided with bioactive agent (hemoglobin having a size of about 64000 Da) dissolved in a solution comprising about 1-2% alginate and a homogenous mixture was prepared. A second container was provided with solution including 0.25% chitosan and 5-50 mM calcium ion in acetate buffer with pH of 4-5. Paraffin oil was added to both containers with controlled stirring to a water-in-oil emulsion. When a suitable, constant emulsion droplet size was obtained, the two containers were merged. As a result, microbeads of a size of about 10-100 μm were formed with a core of alginate polymer with entrapped hemoglobin, having a complex of chitosan-alginate polymer as a surface layer. Calcium ions serve as a crosslinker so as to form pores for entrapment of the bioactive agent. The so formed microbeads are collected and suspended in gelatin (porcine, melting point 40° C.). The resulting suspension was dropped into a gently stirred cooled phase (8-10° C.) of oil (or water). The gelatin formed solid beads in the size of 1 to 2 mm each enclosing a plurality of microbeads (cores) with entrapped bioactive agent. The so formed solid gelatin beads are further processed in a fluidized bed to be coated with an enteric coating as described in previous sections. The described methodology provides an inventive alternative to manufacture beads with a liable bioactive agent that needs to be preserved from excessive loss of water during manufacturing while being safeguarded from structural impairments that may compromise the efficacy of the bioactive agent when delivered to the duodenum. It was also observed that hemoglobin was protected in the microbeads following enzymatic removal with alcase of the gelatin.

1. A pharmaceutical composition for oral or enteral administration comprising beads that admit a bioactive agent, liable to be degraded or inactivated at least when exposed to gastric fluids of the stomach cavity selected from the group of bioactive proteins, antibodies and viable cells, to exert its activity in the duodenum and the upper intestines, wherein the beads comprise at least one ionically cross-linked hydrogel core comprising alginate, b) the bioactive agent entrapped in the hydrogel gel pores, and c) an enteric coating that is water soluble above a threshold pH value between 4 and 7, wherein the water content of the hydrogel is at least 10% of its equilibrium water content.

2. A composition according to claim 1, wherein the water content of the hydrogel is at between 20 to 98%, preferably, between 50 to 98%, more preferably between 70 to 98% of equilibrium water content.

3. A composition according to claim 1, wherein the cores are surrounded with have a gel layer, preferably comprising alginate and chitosan, having pores of smaller average size than the pores of the hydrogel, preferably such pores have a size that sufficiently prevents the bioactive enzymes in the duodenum and the upper intestines to contact and degrade the bioactive agent.

4. A composition according to claim 1, wherein the enteric coating is acylate based preferably, the enteric coating comprising a copolymer of methacrylic acid and an acrylate.

5. A composition according to claim 1, comprising a plurality of enterically coated beads with alginate cores with entrapped bioactive agent having a surrounding layer comprising alginate and chitosan, wherein the beads have an average size in the range of 0.1 to 2 mm.

6. A composition according to claim 1, comprising a plurality of enterically coated beads have an average size in the range of 0.1 to 2 mm, wherein the beads comprises gelatin and alginate cores with entrapped bioactive agent having a surrounding layer comprising alginate and chitosan, wherein the cores have an average size in the range of 10 to 100 μm.

7. A method suitable for manufacturing of bioactive beads according to claim 1, wherein the beads comprise a) an ionically cross-linked hydrogel, b) a bioactive agent entrapped in the gel pores, the method comprising:
i) preparing cores in a solution comprising alginate in the presence of a crosslinking ion and the bioactive active agent;
ii) treating the cores in order to control their water content and structure to thereby settling the bioactive beads ;
iii) fluidizing the beads in a fluidized bed;
iv) feeding a composition comprising an enteric coating agent to the fluidized bed; and
v) collecting the so prepared beads for the preparation of a pharmaceutical composition according to any of claims 1 to 6.

8. The method of claim 7, comprising a step of separating the cores from the solution, wherein step (ii) includes allowing the cores to dry to a controlled water content of at least 10% of the equilibrium water content of the hydrogel, preferably between 20 to 98%, more preferably, between 50 to 98%, still more preferably between 70 to 98% of equilibrium water content.

9. The method according to claim 8, wherein the drying process in step (ii) is performed at ambient temperature for at least one hour, preferably about two hours.

10. The method according to claim 7, wherein step (i) includes preparing the cores in a water-in-oil microemulsion and step (ii) includes adding gelatin to the solution of step (i) at temperature allowing melting of gelatin, forming beads including the cores at a lower temperature and separating the beads.

11. The method of claim 7, wherein the solution of step (i) further comprises chitosan in order to prepare a gel layer of alginate and chitosan surrounding an alginate core with entrapped bioactive agent.

12. The method of claim 7, wherein the step (ii) includes treatment of the cores with a detackifier, such as magnesium stearate or non-crosslinked chitosan; and wherein the composition of step (iv) comprises the enteric coating agent is a solution based on ethanol.

13. A method of treatment comprising intraduodenally inactivating a substance excreted to the duodenum characterized by orally or enterally administering a composition according to claim 1.

14. A method according to claim 13, wherein the administered bioactive agent is an enzyme capable of enzymatically inactivating the substance.
